(19) United States
(12) Patent Application Publication
(10) Pub. No.: US 2021/0329949 A1
(43) Pub. Date: Oct. 28, 2021

(54) PROTEIN-FORTIFIED BEVERAGES FOR ENHANCED ATHLETIC PERFORMANCE

(71) Applicant: Leprino Performance Brands LLC, Denver, CO (US)

(72) Inventors: Jennifer Castro, Highlands Ranch, CO (US); Katie Simons, Denver, CO (US); Bradley Maier, Broomfield, CO (US); Angela Queneau, Denver, CO (US); Guorong Wang, Broomfiled, CO (US); Yukiko Sakai, Tokyo (JP)

(73) Assignee: Leprino Performance Brands LLC, Denver, CO (US)

(21) Appl. No.: 17/238,568

(22) Filed: Apr. 23, 2021

Related U.S. Application Data
(60) Provisional application No. 63/015,973, filed on Apr. 27, 2020.

(51) Int. Cl. A23L 2/66; A23L 33/19; A23L 33/16; A23L 2/68; A23L 2/60; A23L 2/58; A23J 1/20; A23J 3/10; A61K 35/20; A61K 9/00

(52) U.S. Cl. CPC A23L 2/66; A23L 33/19; A23L 33/16; A23L 2/68; A23L 2/60; A23V 2002/00; A23J 1/202; A23J 1/205; A23J 3/10; A61K 35/20; A61K 9/0095; A23L 2/58

(57) ABSTRACT

Protein-containing beverages are described. The beverages may include water and 2 wt. % to 8 wt. % protein (based on the total weight of the beverage). The protein in the beverage may include whey protein. The whey protein has at least 12 wt. % of the branched-chain amino acid leucine (based on the total weight of the whey protein). The beverage may also include casein protein. Methods of making protein-containing beverage products are also described. The methods may include providing a protein-containing aqueous mixture, homogenizing and pasteurizing the protein-containing aqueous mixture, and bottling the homogenized and pasteurized protein-containing aqueous mixture to form the protein-containing beverage product. The protein-containing aqueous mixture may include casein protein, and whey protein having at least 12 wt. % leucine (based on the total weight of the whey protein).

### CROSS-REFERENCE TO RELATED APPLICATIONS

This application claims the benefit of U.S. Application Ser. No. 63/015,973 filed on Apr. 27, 2020, the entire disclosure of which is hereby incorporated by reference for all purposes.

### TECHNICAL FIELD

The technical field is protein-fortified beverages and beverage concentrates for enhanced athletic performance and other purposes. The field also includes methods of making protein-fortified beverages and beverage concentrates. The protein-fortified beverages and beverage concentrates include added proteins that have increased weight percentages of selected essential amino acids (e.g., one or more branched-chain amino acids) relative to the total weight of protein in the beverage. A significant portion of the added proteins may be sourced from native whey protein.

### BACKGROUND

Muscle recovery and hydration are essential processes for enhancing physical performance in a variety of athletic fields including weightlifting, powerlifting, high-intensity interval training, plyometrics, gymnastics, and many other sports and fitness regimens. Muscle recovery includes the consumption of macronutrients, particularly proteins, at frequent intervals that are coordinated with an exercise routine. Hydration includes the steady consumption of water and electrolytes to replace those lost through respiration and perspiration during periods of demanding physical exertion.

Sports beverages are a fast and easy way to supply the water, electrolytes, proteins, and other nutrients before, during, and after intense exercise when an athlete's body is experiencing peak demand. Some sports beverages provide exclusively water and electrolytes, others supply water, electrolytes, and carbohydrates, while still others supply water, carbohydrates and proteins. The water, electrolytes, and carbohydrates in sports beverages primarily serve a replenishment function by supplying these compounds to an athlete who has depleted them, or will deplete them, though intense exercise or a sports competition. The protein in sports beverages primarily serve a muscle recovery and enhancement role by suppling the amino acids that are required for muscle maintenance and growth.

The proteins found in milk are a common source of protein in sports beverages. In fact, bovine milk itself is considered by many sports nutritionists to be the “original” sports drink due to its high concentration of proteins and minerals (i.e., electrolytes). Unfortunately, natural bovine milk also includes high concentrations of dairy fats and lactose sugar that are difficult to digest, especially during intense physical exertion. Thus, many sports beverages include milk proteins that have been separated from other constituents of the milk, including fats and lactose.

One source of milk proteins for sports beverages is the whey protein that is produced as a byproduct of cheese making. During cheesemaking, the casein proteins in the milk are formed into cheese curds while the liquid whey proteins are drained from the curds and diverted to non-cheese uses. In most cheesemaking processes, the initial whey proteins are mixed with a significant amount of lactose and minerals, and the mixture undergoes additional purification to separate the whey proteins from the lactose. Depending on the purification process and the extent of purification, whey protein concentrate (WPC) may be formed that concentrates the whey protein to 25-90 wt. % of protein as a percentage of the total weight of solids, or whey protein isolate (WPI) may be formed that concentrates the whey to 90-99 wt. % of protein as a percentage of the total weight of solids.

Whey proteins derived from cheesemaking also include additional byproducts, such as cheesemaking enzymes and the hydrolyzed proteins they generate. The hydrolyzed proteins include glycomacropeptides (GMPs) that is hydrolyzed from κ-casein so that the resulting para-κ-casein can form a major component of cheese curd. The smaller, more soluble GMPs are carried away with the whey proteins and can constitute 15-20 wt. % of the protein present in the whey protein fraction. Unfortunately, the GMPs are an inferior source of protein for muscle recovery because they have fewer branched chain amino acids that stimulate muscle protein synthesis and are a major building block in muscle tissue during periods of intense exercise and resistance training. In particular, GMPs have lower relative amounts of the amino acid leucine, which is very effective at stimulating muscle protein synthesis, and which has one of highest levels of uptake in muscle tissue during muscle recovery. Research suggests that consumption of proteins with higher than average levels of the amino acid leucine can increase an athlete's ratio of muscle mass to body fat compared to proteins with average or below average levels of leucine. Thus, there is a need for sources of proteins other than whey proteins that were generated as a byproduct of a cheesemaking process. This and other issues are addressed in the present application.

This background section described subject matter related to the present protein-fortified beverages and concentrates, as well as methods of making them. It should not be assumed that anything in this background section constitutes an admission that the above-described subject matter constitutes prior art to the subject matter disclosed below.

### SUMMARY

Protein-fortified beverages are described that incorporate a protein ingredient sourced from a natural protein source that has not been subjected to a process that significantly denatures the protein. These exemplary protein ingredients include concentrated milk proteins such as whey proteins, casein proteins, or both, that are sourced directly from pasteurized skim milk. These whey and/or casein proteins have not been significantly denatured by a process that, for example, acidifies the starting milk, heat treats the starting milk beyond conventional pasteurization, or enzymatically hydrolyzes the milk proteins. For example, the protein ingredient incorporated into the protein-fortified beverage may include native milk proteins (e.g., native whey proteins and/or native casein proteins) that have been concentrated and separated from a starting bovine milk.

The protein ingredient may also be selected to provide increased amounts of proteins that are rich in branched-chain amino acids such as leucine, isoleucine, and valine. These branched-chain amino acids have been shown in sports nutrition studies to be more bioavailable and more readily used in anabolic processes like muscle building than other essential amino acids. The present protein-fortified beverages may incorporate a protein ingredient that is significantly, if not exclusively, weighted with whey proteins that are rich in the branched chain amino acid leucine.

As noted above, the protein-fortified beverage may include a protein ingredient that is also notable for what it does not contain. The protein ingredient may lack one or more compounds that are generated by a process that denatures milk proteins. In some embodiments, the protein ingredient may lack any and all compounds that are generated by any such process. Examples of protein ingredients that fit these criteria include protein ingredients that lack one or more compounds that are generated by a cheesemaking process. These compounds may include compounds produced by enzymatically hydrolyzed casein proteins such as glycomacropeptides (GNPs), para-κ-casein, and enzymatically destabilized casein micelles, among other compounds generated from the enzymatically hydrolyzed casein. The protein ingredient may also lack one or more compounds that facilitate the hydrolysis of the casein proteins, including hydrolysis enzymes that constitute rennet or other coagulants such as chymosin and bovine pepsin; the animal, plant, and fungi sources of rennet (e.g., Aspergillus niger, Kluyveromyces lactis, Mucor mehei, Endothia paracitia, etc.); and compounds produced by cheese starter cultures (e.g., lactic acid, flavors, small peptides, etc.).

The present protein-containing beverages may include water and 2 wt. % to 8 wt. % protein based on the total weight of the beverage. Additional exemplary protein levels in the beverage include a range of 1.7 g to 6.8 g of protein per 100 ml of beverage. The protein may be a combination of proteins that include whey proteins and casein proteins. The whey proteins are rich in branched chain amino acids like leucine. For example, the whey proteins in the present protein-containing beverage may contain at least 12 wt. % leucine based on the total weight of the whey protein. Additional exemplary leucine levels include a range of 2.3 g to 4.0 g of leucine per 2 wt. % to 8 wt. % of protein.

In some embodiments, the whey protein may represent 50 wt. % to 99.9 wt. % of the total protein in the beverage. The casein protein may represent 0.1 wt. % to 50 wt. % of the total protein in the beverage. In some embodiments, the casein protein may include one or more of β-casein, α-s1 casein, α-s2 casein, and κ-casein, sourced directly from milk that has not been used in a cheesemaking process. In still additional embodiments, the proteins in the beverage have a weight ratio of whey proteins to casein proteins that range from 50:50 to 99.9:0.01.

The protein-containing beverage may contain electrolytes (e.g., minerals). These electrolytes may include sodium ions (a.k.a. sodium), potassium ions (a.k.a. potassium), and calcium ions (a.k.a. calcium). among other electrolytes. The electrolyte content may be measured with, for example, atomic emission spectrometry (e.g., ICP-AES) performed on a beverage sample. Exemplary amounts of electrolyte may include ranges of 0.05 wt. % to 0.10 wt % based on the weight of the beverage, and 50 mg to 100 mg per 100 ml of the beverage, among other ranges. The beverage may contain no electrolytes sourced from a cheesemaking process. For example, the beverage may contain no electrolytes incorporated as salts into the milk, curds, and/or cheese during a cheesemaking process. In some cases, the electrolytes contained in the beverage may be entirely derived from milk that remain with the protein following purification and concentration of the proteins from skim milk.

The protein-containing beverage may be low in sodium. For example, the beverage may contain less than 0.03 wt. % sodium based on the total weight of the beverage. Additional exemplary sodium levels in the beverage include a range of 10 mg to 100 mg of sodium per 100 ml of beverage. The beverage may contain no sodium derived from salt (e.g., sodium chloride) added to the beverage's proteins. Exemplary sources of sodium in the protein-containing beverage may include sodium citrate that is incorporated into the beverage. Similarly, in some embodiments the protein-containing beverage may include no added chloride ions. These include embodiments where the beverage includes no added chloride ions derived from salt added to the beverage's proteins.

The protein-containing beverage may contain potassium. The protein-containing beverage may include no added potassium beyond the potassium found in the beverage's proteins. For example, the beverage may contain potassium at a level of 0.02 wt. % to 0.3 wt. % based on the total weight of the beverage. Additional exemplary potassium levels in the beverage include a range of 8 mg to 100 mg of potassium per 100 ml of beverage. The beverage may contain potassium derived from a potassium compound (e.g., a potassium salt) added to the beverage's proteins.

The protein-containing beverage may include no added calcium beyond the calcium found in the beverage's proteins. The calcium in the protein-containing beverage may be at a level of 0.14 wt. % to 1.65 wt. % based on the total weight of the beverage. Additional exemplary calcium levels in the beverage include a range of 8 mg to 350 mg of calcium per 100 ml of beverage. In additional embodiments, one or more calcium salts may be added to the beverage.

The protein-containing beverage may contain little or no lactose (e.g., less than 1 g of lactose per 100 ml of beverage). Lactose is a sugar typically found in bovine milk at levels of about 4.5 wt. % to 5 wt. % by dry weight. Lactose has a relatively low sweetness level compared to sucrose, and is difficult for many people to digest, especially as they age and lose a greater portion of their lactose hydrolysis enzymes. Most common varieties of cheese have significantly lower levels of lactose than the milk used to make them, but much of that lactose is incorporated into the protein byproducts (e.g., cheese whey) that is separated from the curds used to make the cheese. Embodiments of the protein-containing beverage may include lactose levels ranging from 0 wt. % to 2 wt. % based on the total weight of the beverage. In some embodiments, the lactose levels in the beverage may be below the limits of detectability with conventional equipment, and therefore essentially 0 wt. % based on the weight of the beverage.

Embodiments of the protein-containing beverage lack one or more ingredients found in proteins that have been sourced from a cheesemaking process. These ingredients include casein hydrolysis enzymes such as rennet, fungal and bacterial sources of the casein hydrolysis enzymes, culture mediums used to grow cheese starter cultures, hydrolyzed casein proteins including glycomacropeptides (GMPs), whey proteins derived by separation from curd particles or a curd coagulum, and lactic acid, among other cheesemaking ingredients. In some embodiments, the protein-containing beverage lacks casein hydrolysis enzymes and the bacterial or fungal sources of those enzymes, as well as the starter culture medium for cheese starter bacteria. In still further embodiments, the protein-containing beverage lacks lactic acid.

The protein-containing beverage may be acidic (e.g., a pH of 4.6 or less). Exemplary pH ranges for the beverage include 2.5 to 4.5; 3.0 to 4.0; and 3.2 to 3.6, among other pH ranges. The pH may be measured by titration of the beverage with a standard base to provide the total acid concentration of the beverage. The beverage may have an acidic pH with no precipitation of the beverage's proteins. For example, the beverage may have a pH of 4.6 or less and have no perceptible casein precipitates. The pH of the beverage mixture may be adjusted by the incorporation of one or more food-grade acids. Exemplary acids include phosphoric acid, malic acid, gluconic acid, and citric acid, among other acids.

The protein-containing beverage may have a fluidity and mouthfeel similar to water. Exemplary viscosities of the protein-containing beverage may range from 3 cP to 10 cP at temperatures ranging from 5° C. to 23° C. (i.e., room temperature). Embodiments of the protein-containing beverage may have all the solids fully dissolved in water to create a water-white aqueous solution. Alternatively, the protein-containing beverage may have one or more ingredients that are less than fully dissolved in the water to create a colloidal suspension or mixture that can develop sediment. In these instances, the packaging on the protein-containing beverage may include an instruction to “shake well” before drinking.

Methods of making protein-containing beverages are also described. These methods may include filtering milk to form a protein isolate. The protein isolate may include whey proteins and casein proteins. The proteins may include at least 12 wt. % leucine based on the total weight of the whey proteins. The method may further include combining the protein isolate with an aqueous composition to form the protein-containing beverage. In some embodiments, the protein isolate contains 50 wt. % to 99.9 wt. % whey proteins. In further embodiments, the protein isolate contains 0.1 wt. % to 50 wt. % casein proteins.

Additional methods of making protein-containing beverages are described. These methods may include providing a protein-containing aqueous mixture that includes total proteins comprising casein proteins and whey proteins. The proteins may include at least 12 wt. % leucine based on the total weight of the whey proteins. The method may further include homogenizing and pasteurizing the protein-containing aqueous mixture. The homogenized and pasteurized protein-containing mixture may be bottled to form the final protein-containing beverage. These additional methods include embodiments where the pH of the protein-containing beverage is adjusted to a range of 3 to 4 (e.g., 3.5) by incorporation of a food-grade acid.

The present methods of making protein-containing beverages may also include measuring a content of one or more branched chain amino acids such as leucine in the protein-containing mixture. The methods may further include adjusting the content of one or more branched-chain amino acids (e.g., leucine) to a level of 12 wt. % or more as a function of the total weight of whey protein in the beverage. In some embodiments, this adjustment in the branched-chain amino acid content does not include adding free amino acids to the protein-containing beverages.

The above described protein-containing beverages and methods of making them may be further concentrated for packing and shipping. In some embodiments, these protein-containing beverage concentrates may have 5 wt. % to 95 wt. % of the water removed before packaging and shipping of the protein-containing beverage concentrate. In additional embodiments, the concentrate is initially formed by using less water in the ingredients used to make the beverage. For example, a protein-containing aqueous mixture used to make the beverage may be provided with substantially less water than is included in the final protein-containing beverage. In some embodiments, the concentrate is an aqueous solution, while in other embodiments the concentrate is an aqueous suspension or slurry. In still further embodiments, enough water may be removed to convert the protein-containing beverage into a powdered mixture that may be rehydrated into the beverage when the consumer is ready to drink it. Unless otherwise specified, the term protein-containing beverage also encompasses concentrates of the beverage.

### BRIEF DESCRIPTION OF THE DRAWINGS

A further understanding of the nature and advantages of the disclosed embodiments may be realized by reference to the remaining portions of the specification and the drawings.

FIG. 1 shows a flowchart with selected steps in a method of making a protein-containing beverage according to present embodiments.

FIG. 2 shows a capillary electrophoresis profile of native whey protein isolate.

FIG. 3 shows a capillary electrophoresis plot that overlays the protein profile of a native whey protein with two protein profiles for whey proteins derived from cheesemaking processes.

FIG. 4 shows a capillary electrophoresis profile of whey protein isolate derived from whey generated by a conventional cheesemaking process.

FIG. 5 shows a capillary electrophoresis profile of an exemplary protein-containing beverage according to an inventive embodiment.

FIG. 6 shows a capillary electrophoresis profile of a first, conventional protein-water beverage.

FIG. 7 shows a capillary electrophoresis profile of a second, conventional protein-water beverage.

FIG. 8 shows a capillary electrophoresis profile of a third, conventional protein-water beverage.

Several of the figures are included as schematics. It is to be understood that the figures are for illustrative purposes, and are not to be considered of scale unless specifically stated to be of scale. Additionally, as schematics, the figures are provided to aid comprehension and may not include all aspects or information compared to realistic representations, and may include exaggerated material for illustrative purposes.

In the figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION

Protein-containing beverages are described that incorporate a protein ingredient that includes one or more sources of natural, protein. In some embodiments, these natural, proteins are the exclusive sources of protein incorporated into the beverage. Exemplary protein ingredients include undenatured milk proteins sourced directly from pasteurized or unpasteurized, starting bovine milk. These undenatured milk proteins may include native whey proteins, native casein proteins, or a combination of both types of proteins. In some embodiments, the one or more of the native whey proteins may have a concentration profile that is the same as, or similar to, their concentration profile in an unprocessed, starting bovine milk. In additional embodiments, the native whey proteins may have a different concentration profile than their concentration profile in the unprocessed, starting bovine milk. In some embodiments, the concentration profiles of one or more native casein proteins may also be similar to, or the same as, their concentration profiles in the starting bovine milk. In additional embodiments, the concentration profiles of one or more native casein proteins may be different from their concentration profiles in the starting bovine milk.

The protein ingredient incorporated into the protein-containing beverages may be selected to include proteins that are rich in the amino acid leucine and other branched-chain amino acids (e.g., isoleucine, valine). The protein ingredient may include whey proteins that include a minimum threshold level of branched-chain amino acids that is characteristic for native whey proteins. For example, the protein-containing beverages may include whey proteins that have at least 5 wt. % leucine (a branched-chain amino acid) based on the total weight of the whey proteins. Other exemplary threshold levels for the leucine content of the whey protein in the beverage include at least 12 wt. %, at least 13 wt. %, at least 14 wt. %, at least 15 wt. %, at least 16 wt. %, at least 17 wt. %, at least 18 wt. %, at least 19 wt. %, at least 20 wt. %, etc., based on the total weight of the whey proteins. Exemplary ranges for the weight percentage of leucine per total weight of the whey proteins include 5 wt. % to 20 wt. %; 10 wt. % to 20 wt. %; 10 wt. % to 18 wt. %; 11 wt. % to 20 wt. %; 11 wt. % to 16 wt %; 12 wt. % to 14 wt. %; among other weight ranges.

The protein ingredient incorporated into the protein-containing beverages may be selected to include proteins that are rich in essential amino acids other than the above-described branched-chain amino acids. These essential amino acids include histidine, lysine, methionine, phenylalanine, threonine, and tryptophan. For example, the protein-containing beverages may include proteins that have at least 3 wt. % essential amino acids selected from histidine, lysine, methionine, phenylalanine, threonine, and tryptophan. Exemplary ranges for these essential amino acids may also include 3 wt. % to 20 wt. % of the total proteins in the protein-containing beverage. Additional exemplary ranges include 5 wt. % to 15 wt. %, 5 wt. % to 12.5 wt. %, 5 wt. % to 10 wt. %, and 5 wt. % to 7.5 wt. %, among other exemplary ranges for the essential amino acids. In further examples, one or more of the above listed essential amino acids may be present in an amount ranging from 1 wt. % to 10 wt. % based on the total weight of the proteins in the protein-containing beverage. For example, the essential amino acid threonine may be present in 1 wt. % to 10 wt. %, 2 wt. % to 8 wt. %, 3 wt. % to 6 wt. %, etc., based on the total weight of the proteins in the protein-containing beverage. These exemplary ranges may also apply to one of the other essential amino acids.

The protein-containing beverage may also include combinations of the above-described branched-chain amino acids and the above-described essential amino acids. For example, the protein-containing beverage may include 5 wt. % or more of at least one branched-chain amino acid (e.g., 5 wt. % to 20 wt. %, 11 wt. % to 18 wt. %, etc.) and 5 wt. % or more of at least one of the above-described essential amino acids (e.g., 5 wt. % to 20 wt. %, 3 wt. % to 10 wt. %, 5 wt. % to 12 wt. %, etc.).

In several embodiments, some or all of the proteins included in the protein ingredient of the beverage have not been subjected to any processes that involve denaturing the proteins. These denaturing processes include processes that significantly alter the shape of a native protein, processes that fragment the native protein by enzymatic and/or chemical hydrolysis, and processes that significantly alter one or more ionic characteristic of the native protein (e.g., permanently altering its isoelectric point, etc.), among other processes. However, the protein ingredient may include native proteins that have been concentrated or isolated from an unprocessed, natural protein source such as a starting bovine milk. Such processes may include evaporative processes that remove water from the milk, filtration processes (e.g., microfiltration, diafiltration, ultrafiltration, nanofiltration, etc.) that separate the constituents of the milk into permeate and retentate fractions, chromatography processes (e.g., column chromatography) that capture targeted constituents of the milk on a substrate while permitting others to pass in an effluent, precipitation processes, and dialysis processes, among other processes.

As noted above, the proteins in the protein ingredient of the present beverages may lack any proteins sourced from a cheesemaking process, such as whey proteins separated as a byproduct during the formation, cooking, and/or mechanical working of cheese curds. In most instances, the whey protein byproducts have been (i) denatured due to cheesemaking conditions such as enzymatic hydrolysis, chemical acidity, and/or high temperatures, (ii) contaminated with other cheesemaking compounds such as starter cultures, hydrolysis enzymes, acids, protein fragments, surfactants, emulsifiers, etc. In some examples, the cheese whey proteins may be fractioned by filtration or chromatography processes, and as a result they do not contain all the primary whey proteins, including α-lactalbumin, β-lactoglobulin, or glycomacropeptides. In contrast, native whey proteins obtained directly from bovine milk that has not been used in cheesemaking retain the undenatured whey proteins found in the starting milk (e.g., -lactalbumin, β-lactoglobulin, etc.) and lack compounds generated by the cheesemaking (e.g., glycomacropeptides, lactic acid, starter cultures, etc.). The native whey proteins may also include soluble casein proteins, such as one or more of β-casein, α-casein, and κ-casein. These differences are apparent even when the undenatured whey proteins are concentrated and/or isolated from the starting bovine milk by removal of most casein proteins as well as the milk's non-protein constituents including dairy fats, lactose, and minerals. These concentration/isolation processes of obtaining the native whey proteins from milk all share common characteristics of keeping the separated whey proteins intact (i.e., not hydrolyzed), undenatured by exposure to high temperatures or extreme pH, and uncontaminated with hydrolysis products of casein proteins that were formed during a cheesemaking process.

As noted above, the protein ingredient in the protein-containing beverage may include native whey proteins that have not been significantly denatured from their natural state in unprocessed bovine milk. Native whey proteins may also be referred to as serum whey proteins, ideal whey proteins, milk-whey proteins, milk-derived whey proteins, milk-soluble whey proteins, milk-serum whey proteins, casein-reduced milk proteins, and casein-depleted milk proteins, among other names. However, it should not be assumed that all whey proteins labeled with these names are actually native whey proteins. The native whey proteins in the starting milk have not been exposed to one or more denaturing steps such as hydrolysis enzymes, high temperatures (e.g., temperatures greater than 74° C.), etc., that can denature the native whey proteins. Examples of these whey protein denaturing steps can be found in processes such as cheesemaking, yogurt making, the production of dairy gels, and the production of dairy powders, among other processes that use unprocessed bovine milk as a starting ingredient. When one or more whey proteins in the unprocessed, starting bovine milk has been significantly denatured (e.g., significantly altered in shape, fragmented by hydrolysis, significantly altered in one or more ionic characteristic such as isoelectric point, etc.) it is no longer a native whey protein even if labeled as such. Additional details about the protein-containing beverages and methods of making them are now discussed.

### Exemplary Protein-Containing Beverages

An exemplary protein-containing beverage may include water, protein, one or more carbohydrates, one or more acidification agents, and one or more flavoring agents. In additional embodiments, the protein-containing beverage may include one or more of a probiotic, a botanical, a fruit ingredient, a vegetable ingredient, caffeine, and collagen. The water may constitute 90 wt. % to 97 wt. % (e.g., 94 wt. % to 96 wt. %) of the total weight of the beverage. The protein may constitute 2 wt. % to 8 wt. % (e.g., 3.5 wt. % to 5.5 wt. %) of the total weight of the beverage. The one or more carbohydrates may constitute 0.01 wt. % to 2 wt. % (e.g. about 1 wt. %) of the total weight of the beverage. The one or more acidification agents and one or more flavoring agents may together constitute 0.1 wt. % to 1 wt. % of the total weight of the beverage. The beverage may include sodium, potassium, and calcium supplied by the above-listed ingredients. For example, the beverage may include 0.01 wt. % to 0.1 wt. % (e.g., 0.02 wt % to 0.05 wt. %) sodium, 0.02 wt. % to 0.3 wt. % (e.g., 0.02 wt. % to 0.1 wt. %) potassium, and 0.04 wt. % to 1.6 wt. % (e.g., 0.05 wt. % to 1 wt. %) calcium.

The exemplary protein-containing beverage may include proteins sourced directly from bovine milk that have not been denatured by a thermal, acidification, or enzymatic process. For example, the proteins may exclude whey proteins denatured by excessive heating. In some instances these thermally denatured whey proteins have formed complexes with casein proteins. The proteins may also exclude casein proteins that have been chemically aggregated by placing them in an acidic environment (e.g., excessive levels of lactic acid and/or hydrochloric acid), or enzymatically hydrolyzed casein proteins and their protein hydrolysates (e.g., glycomacropeptides) generated by cheesemaking processes.

The protein in the beverage may include whey proteins that have been concentrated directly from bovine milk by separating the whey proteins from at least a portion of the milk's water, fats, other proteins (e.g. casein proteins), sugars (e.g., lactose), and minerals, among other milk ingredients. For example, the whey proteins may be sourced from a nonfat milk where the milk fats have been reduced from a starting amount of approximately 3.25 wt. % to less than 1 wt. % (based on the total weight of the milk). The nonfat milk may be subjected to one or more filtration steps (e.g., microfiltration, ultrafiltration, diafiltration) that separates the whey proteins from the majority of the milk's minerals, sugars, and casein proteins without first aggregating or gelling the casein protein. In some instances, the filtered whey proteins may be further treated by a delactosing process to remove more of the milk sugar (e.g., lactose).

The protein ingredient produced by these separation and filtration processes may include 50 wt. % or more proteins based on the total weight of the protein ingredient. In some embodiments, the protein ingredient may be a whey protein concentrate having 50 wt. % to 89.5 wt. % proteins based on the total weight of the protein ingredient. In additional embodiments, the protein ingredient may be a whey protein isolate having greater than 89.5 wt. % (e.g., 90 wt. % to 99.5 wt. %) proteins based on the total weight of the protein ingredient. The protein ingredient may include both whey proteins and casein proteins. Embodiments include a weight ratio of whey proteins to casein proteins (i.e., WP to CN) ranging from 50:50 to 99.9:0.1. In these embodiments, the whey proteins may constitute 50 wt. % to 99.9 wt. % of the total proteins in the protein ingredient. Table 1 below lists the relative amount of β-casein as a weight percentage of (i) the total protein, and (ii) the total casein protein, present in the beverage for eight exemplary weight ratios of whey proteins to casein proteins:

TABLE 1
Weight Percentages of β-Casein for
Various Ratios of Whey-To-Casein Proteins:

| Weight Ratio of Whey | Weight Percentage | Weight Percentage |
|---|---|---|
| Protein to Casein | β-CN to | β-CN to All |
| Protein (WP:CN) | Total Protein (%) | Casein Proteins (%) |
| 95:5 | 4% | 42% |
| 90:10 | 7% | 45% |
| 80:20 | 9% | 50% |
| 75:25 | 15% | 72% |
| 70:30 | 22% | 75% |
| 60:40 | 31% | 78.5% |
| 55:45 | 36% | 84% |
| 50:50 | 45.5% | 91% |

The casein and whey proteins found in bovine milk are actually groups of proteins: In starting bovine milk the casein proteins may include αₛ₁-casein (30-42 wt. % based on total weight of proteins in the milk), β-casein (25-35 wt. %), κ-casein (8-13 wt. %), and αₛ₂-casein (8-13 wt. %). The whey proteins in staring bovine milk may include β-lactoglobulin (5-15 wt. % based on total weight of proteins in the milk), α-lactalbumin (1.5-5 wt. %), immunoglobulins (Igs) (1-3 wt. %), bovine serum albumin (BSA) (0.2-0.5 wt. %), and proteose peptones (1.5-7 wt. %).

The relative amounts of casein and whey proteins in the protein ingredient incorporated into the protein-containing beverage may be significantly changed from an unprocessed, starting bovine milk: as noted above, the filtration of the starting milk causes the permeation of many whey proteins through a membrane while holding back many of the casein proteins in a retentate. The permeate has a reverse weight ratio of whey proteins to casein proteins from what is found in the starting bovine milk (e.g., about 80 wt. % casein proteins and about 20 wt. % whey proteins in starting milk). On the other hand, the concentration profile of the casein proteins (i.e., αₛ₁-casein, β-casein, κ-casein, and αₛ₂-casein) may be approximately the same as that measured in unprocessed bovine milk since the milk has not been subjected to thermal, acidification, or enzymatic processes like cheesemaking. In additional embodiments, one or more of the casein proteins may have a concentration relative to one or more other casein proteins that differs from their relative concentrations in an unprocessed, starting bovine milk. For example, the casein proteins in the protein ingredient incorporated into the beverage may be more concentrated in β-casein (e.g., 40 wt. % to 95 wt. % based on the total weight of the casein proteins) than measured in unprocessed bovine milk. The concentration differences may be caused by one or more processes that are used to separate the milk components without denaturing the casein proteins (e.g., filtration). Both the whey and casein proteins may remain in solution over the entire pH range of the protein-containing beverage. The proteins remain in solution even when the protein-containing beverage reaches and falls below the isoelectric point of the casein proteins (i.e., pH 4.6). The absence of casein protein precipitates at pH 4.6 or less is unexpected, since casein proteins typically precipitate out of aqueous solution in this acidic pH range. The protein solubility of the beverage may be measured by quantifying the amount of protein left in the supernatant fraction of a centrifuged sample of the beverage. The higher the protein content in the supernatant, the higher the solubility of the protein in the beverage. On the other hand, higher the protein content in the solid pellet at the end of the centrifuge tube, indicates lower a solubility of the protein in the beverage.

Concentration profiles of the different whey proteins (e.g., the relative weight ratio of β-lactoglobulin to α-lactalbumin) in the protein ingredient incorporated into the beverage may also be similar to those measured in the starting milk. For example the β-lactoglobulin to α-lactalbumin weight ratio in the protein ingredient may have a range of 2:1 to 4:1 (e.g., 3:1), which parallels the β-lactoglobulin to α-lactalbumin weight ratio in unprocessed bovine milk. Similarly, the protein ingredient may have concentrations of one or more other whey proteins (e.g., Igs, BSA, proteose peptones, etc.) that parallel the concentrations of these whey proteins in unprocessed bovine milk. In additional embodiments, one or more of the whey proteins may be reduced or removed from the protein ingredient incorporated into the beverage. For example, the whey protein profile of the protein ingredient may have significantly reduced levels of β-lactoglobulin or α-lactalbumin compared to unprocessed bovine milk. Embodiments of these protein ingredients include α-lactalbumin-depleted protein ingredients having a β-lactoglobulin to α-lactalbumin weight ratio of 1000:1 to 5:1 (e.g., 100:1 to 10:1). Embodiments of these protein ingredients also include β-lactoglobulin-depleted protein ingredients having a β-lactoglobulin to α-lactalbumin weight ratio of 1:1000 to 1:5 (e.g., 1:100 to 1:10). The concentration differences may be caused by one or more processes that are used to separate the milk components without denaturing the whey proteins (e.g., filtration).

The exemplary protein-containing beverage may also have a number of organoleptic qualities that make the beverage desirable to drink. These organoleptic qualities include the viscosity of the beverage, the texture (e.g., graininess) chalkiness of the beverage, the turbidity of the beverage, the acidity of the beverage, the color of the beverage, the flavor of the beverage (e.g., sweetness), and the transparency of the beverage, among other organoleptic qualities. For example, the turbidity of the protein-containing beverage indicates the degree of cloudiness (e.g., opacity) of the beverage, and may be measured by the amount of light scattered by the particles suspended in the beverage. Viscosity characterizes the fluid thickness of the beverage, and may be measured by changes in rotation speed of a spindle with a defined rotation parameter using an instrument such as a Brookfield viscometer. In some embodiments, the beverage is sweetened without the addition of conventional sweeteners such as sucrose, stevia, and/or sucralose, among other conventional sweeteners.

### Exemplary Methods of Making Protein-Containing Beverages

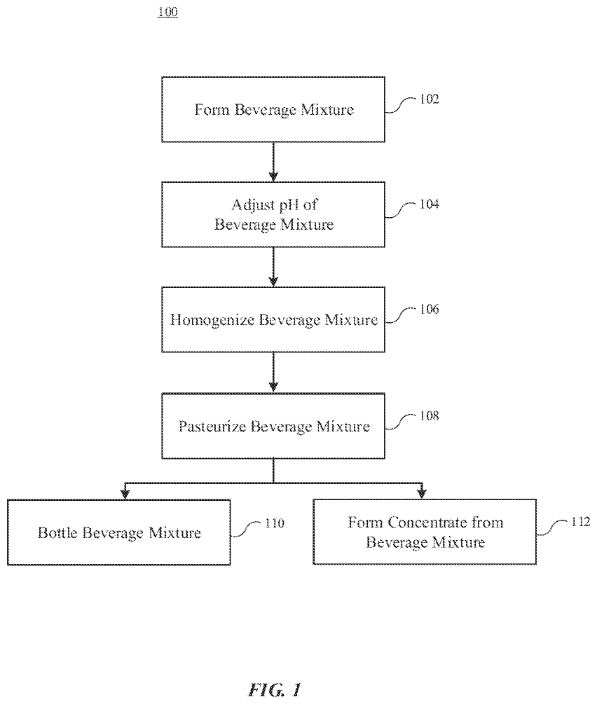

FIG. 1 shows selective aspects of an example of a method 100 of making the present protein-containing beverages. The method 100 includes combining water and a protein ingredient to form an aqueous protein mixture 102. The method may further include combining additional ingredients with the aqueous protein mixture to form an intermediate beverage mixture. These additional ingredients may include one or more of flavor agents, color agents, and sweetening agents, among other types of ingredients. The intermediate beverage mixture may be stirred for a predetermined period (e.g., 10-60 minutes) to permit that protein ingredient and additional ingredients time to hydrate. An edible acid (e.g., phosphoric acid) may be added to the hydrated intermediate beverage mixture to adjust the pH of the mixture to a target level (e.g., pH between 2.5 and 4; pH of 3.5, etc.) 104. The acidified, hydrated intermediate beverage mixture may be homogenized to form a homogenized beverage mixture 106. Homogenization of the acidified, hydrated intermediate beverage mixture may be conducted in a two-stage homogenization process that includes a first, higher-pressure stage (e.g., 2000 psi) and a second, lower-pressure stage (e.g., 500 psi). The homogenized beverage mixture may be pasteurized 108. Exemplary pasteurization conditions may include pasteurization temperatures ranging from 190° F. to 220° F. and pasteurization times ranging from 5-60 seconds (e.g., 6 seconds). The pasteurized, protein-containing beverage may be bottled to form the final, protein-containing beverage 110. The pasteurized, protein-containing beverage may be injected into the bottles at a hot temperature (e.g., 175-193° F.) with the bottling stage marking the end of the pasteurization of the protein-containing beverage. In some embodiments, an orifice of the bottle representing a critical control point (CCP) may be kept at a temperature greater than 175° F. while the bottle is being filled to prevent the introduction of food spoilage microorganisms and/or other contaminants. In other embodiments the protein-containing beverage may be concentrated for packaging and shipping as a reduced-water concentrate 112.

Additional examples of methods of making the present protein-containing beverages (not shown) may include acidifying the protein ingredient to form an acidified protein mixture. Acidification may be done by adding an edible acid (e.g., phosphoric acid) to the acidified protein mixture to adjust the pH of the mixture to a target level (e.g., pH between 2.5 and 4; pH of 3.5, etc.). Additional ingredients may be acidified with the protein, or added to the acidified protein mixture. These additional ingredients may include one or more of flavor agents, color agents, and sweetening agents, among other types of ingredients. Water may be added to the acidified protein mixture, and the mixture may be stirred for a predetermined period (e.g., 10-60 minutes) to give the ingredients time to hydrate. The acidified, hydrated intermediate beverage mixture may be homogenized to form a homogenized beverage mixture 106. Homogenization of the acidified, hydrated intermediate beverage mixture may be conducted in a one or two-stage homogenization process. The two-stage homogenization process may include a higher-pressure stage (e.g., 2000 psi) and a lower-pressure stage (e.g., 500 psi). In some examples of the two-stage homogenization process, the higher-pressure stage is performed first, while in other examples, the lower-pressure stage is performed first. The intermediate beverage mixture may be pasteurized. In some examples, the intermediate beverage is pasteurized before homogenization. In some examples, the intermediate beverage is pasteurized after homogenization. In still further examples, the intermediate beverage is pasteurized and homogenized at the same time. Exemplary pasteurization conditions may include pasteurization temperatures ranging from 190° F. to 220° F. and pasteurization times ranging from 5-60 seconds (e.g., 6 seconds). The pasteurized, protein-containing beverage may be bottled to form the final, protein-containing beverage. The pasteurized, protein-containing beverage may be injected into the bottles at a hot temperature (e.g., 175-193° F.) with the bottling stage marking the end of the pasteurization of the protein-containing beverage. In some embodiments, an orifice of the bottle representing a critical control point (CCP) may be kept at a temperature greater than 170° F. while the bottle is being filled to prevent the introduction of food spoilage microorganisms and/or other contaminants. In other embodiments the protein-containing beverage may be concentrated for packaging and shipping as a reduced-water concentrate.

### Experimental

### Amino Acid Profiles for Various “High-Protein” Foods

Protein profiles for a number of “high-protein” foods were compared with the protein profile of an embodiment of the present protein-containing beverages. Protein content can be measured by a classical Kjeldhal methodology using calibrated and automated instrumentation. Table 2 below list the weight percentage of branched-chain amino acids, and specifically leucine, in serving of each food that has 20 grams of total protein:

TABLE 2
Amino Acid Profile of Food Servings That Have 20 Grams Total Protein:

| | Protein- | Soy | Pea | | | | Hard | |
|---|---|---|---|---|---|---|---|---|
| | Containing | Protein | Protein | Rice | | | Boiled | Alaskan |
| | Beverage | Isolate | Isolate | Protein | Chicken | Beef | Egg | Salmon |
| Essential | 9.1 | 7.7 | 7.9 | 7.6 | 8.6 | 8.4 | 8.9 | 8.6 |
| Amino Acids | | | | | | | | |
| Branched- | 22.8 | 3.6 | 3.7 | 3.8 | 3.7 | 3.7 | 4.0 | 3.4 |
| Chain Amino | | | | | | | | |
| Acids | | | | | | | | |
| Leucine | 12.3 | 1.6 | 1.7 | 1.7 | 1.7 | 1.7 | 1.8 | 1.7 |

As Table 2 demonstrates, the present protein-containing beverage has significantly higher percentages of branched-chain amino acids such as leucine compared with the other listed protein sources of soy protein isolate, pea protein isolate, rice protein, chicken, beef, hard-boiled eggs, and Alaskan salmon. The protein-containing beverage had approximately 140-148% higher percentage of branched-chain amino acids than the other protein sources, and a 149-154% higher percentage of the BCAA leucine. As noted above, branched-chain amino acids (in particular leucine) have been shown in sports nutrition studies to be more bioavailable and more readily used in anabolic processes like muscle building than other essential amino acids.

### Comparison of Native Whey Protein and Cheese Whey Protein

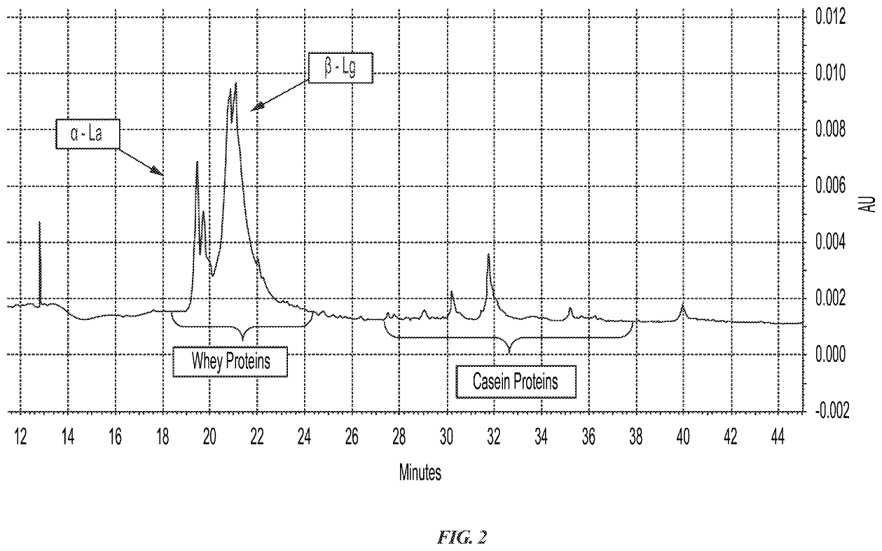

The protein-containing beverage listed in Table 2 above includes native whey protein as the protein ingredient. The protein profile of the native whey protein, and other proteins, can be measured using capillary electrophoresis. Capillary electrophoresis works by separating the proteins based on their charge and size and detecting the relative amounts of separated proteins with the aid of diode-array detection (DAD). Because whey and casein proteins have different sizes and charges, they can be separated and measured using this capillary electrophoresis technique. A capillary electrophoresis profile of the native whey protein is shown in FIG. 2. The profile shows large peaks for the whey proteins α-lactalbumin and β-lactoglobulin, and significantly smaller peaks for the casein proteins. No discernable peak is seen for casein-glycomacropeptides (cGMPs). This protein profile is consistent for a native whey protein derived by filtration of undenatured dairy milk. The filtration process passes through the smaller whey proteins (e.g., α-lactalbumin and β-lactoglobulin) in the filtration permeate while capturing micellar casein in the filtration retentate. As discussed below, some of the casein proteins that are not incorporated into the larger casein micelles are passed through with the whey proteins in the permeate. The largest fraction of these smaller casein proteins is β-casein (β-CN).

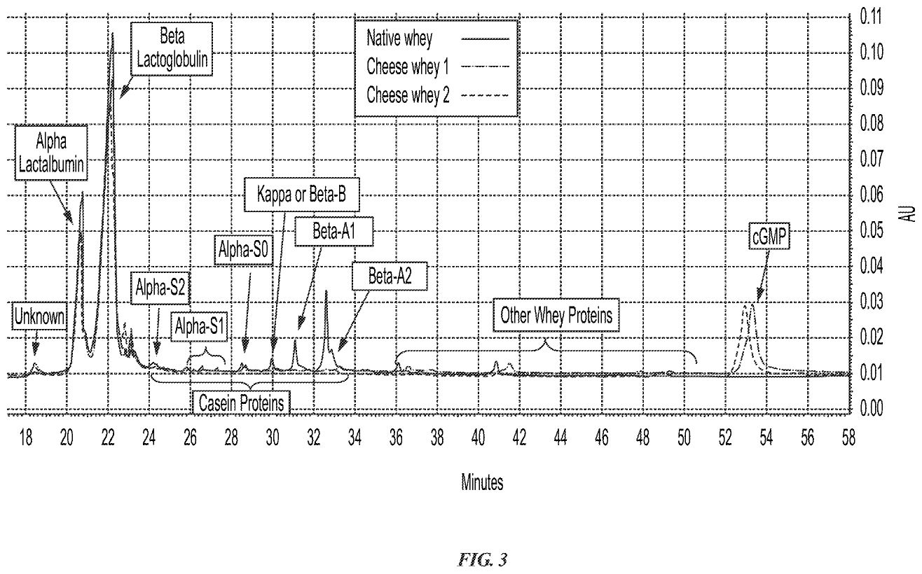

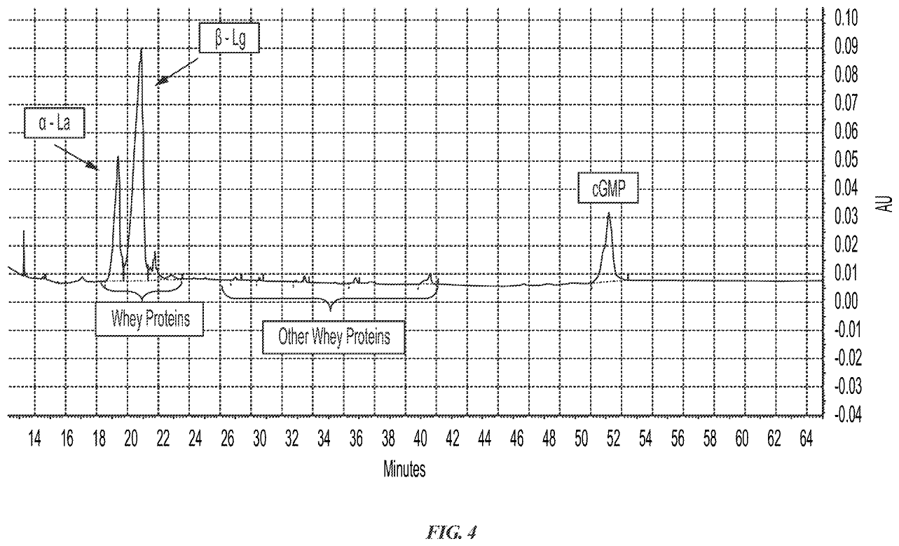

A contrast in the protein profiles can be seen when comparing the native whey protein profile in FIG. 2 with the profiles of whey protein derived from cheesemaking processes that are shown in FIGS. 3 and 4. FIG. 3 show an overlap of three capillary-electrophoresis protein profiles that include a native whey protein profile and two whey protein profiles that have whey proteins derived from cheesemaking processes. FIG. 4 shows capillary electrophoresis protein profile for a whey protein isolate (WPI) derived for a cheesemaking process. The native whey protein profile shown in FIG. 3 has a larger peak for the whey proteins α-lactalbumin and β-lactoglobulin than either of the whey protein sources from cheesemaking (i.e., “Cheese Whey” 1 and 2). The native whey protein profile also shows significant peaks for smaller casein proteins (e.g., α, β, and κ-caseins) that are not detectable in the protein profiles for Cheese Whey 1 and 2. This is explained by the greatly depleted amounts of all casein proteins in Cheese Whey 1 and 2 that were formed into cheese curds during cheese making processes. On the other hand, Cheese Whey 1 and 2 both have significant peaks for the smaller cGMP fragments that are also generated during cheesemaking. The native whey protein profile has no discernable cGMP peak.

The protein profile shown in FIG. 4 shows a similar pattern to Cheese Whey 1 and 2 in FIG. 3, only with larger peaks for the α-lactalbumin and β-lactoglobulin whey proteins relative to the cGMP peak. This is because the WPI undergoes more processing to separate the whey proteins from other proteins in the cheese whey like cGMPs. Both types of whey protein sourced from cheesemaking processes produce no discernable peaks for intact casein proteins.

The native whey protein profile shown in FIG. 2 indicates there are no cGMPs present in the sample. However, the peaks for the casein proteins indicate about 13 wt. % of the total proteins are intact casein proteins, with the largest portion being β-casein. In contrast, the cheese whey protein profiles in FIGS. 3 and 4 indicate about 14 wt. % of the proteins are cGMPs, and less than 2 wt. % are intact casein proteins. The differences in the protein profiles between native whey protein and cheese whey protein produce differences in the amino acid profiles of the protein samples. Table 3 below shows the amino acid profiles of the native whey protein samples having larger weight percentages of branched-chain amino acids and other essential amino acids than the amino acid profiles of whey protein isolate derived from cheese whey:

TABLE 3
Amino Acid Profiles for Native
WPI and Cheese-Whey-Derived WPI

| Amino Acid | | Cheese-Whey- |
|---|---|---|
| (g/100 g of protein) | Native WPI | Derived WPI |
| Alanine | 4.11 | 4.47 |
| Arginine | 2.54 | 1.94 |
| Aspartic acid | 10.84 | 11.08 |
| Cystine | 2.69 | 2.64 |
| Glutamic acid | 17.32 | 17.65 |
| Glycine | 1.57 | 1.41 |
| Histidine | 2.13 | 1.67 |
| Isoleucine | 5.54 | 6.67 |
| Leucine | 12.02 | 10.23 |
| Lysine | 9.96 | 9.59 |
| Methionine | 2.28 | 2.21 |
| Phenylalanine | 3.83 | 3.03 |
| Proline | 5.03 | 5.67 |
| Serine | 3.83 | 4.25 |
| Threonine | 4.60 | 6.87 |
| Tryptophan | 2.79 | 1.98 |
| Tyrosine | 3.65 | 2.97 |
| Valine | 5.29 | 5.66 |
| Essential Amino Acids | 52.07 | 50.88 |

Table 3 shows that the whey protein isolate sample derived from native whey protein had an increased weight percentage of essential amino acids, and in particular an increased weight percentage of leucine, compared to the whey protein isolate sample derived from whey sourced from a cheese making process (i.e., cheese-whey derived WPI).

The differences in the protein profiles between native whey protein and cheese whey protein also affect the physical characteristics of the protein samples. For example, the significant amount of intact casein proteins in native whey protein samples have been shown to impart greater heat stability to beverages made with those samples compared to beverages made with cheese whey protein samples. Heat stability tests were run on test beverages made with (i) 5% w/w native whey protein isolate in water (see FIG. 2 above), and (ii) 5% w/w cheese whey protein isolates in water (see FIG. 3 above). The test beverages were heated treated in an oil bath that held the temperature of the beverage at 90.5° C. for 20 minutes and the results are listed in Table 4:

TABLE 4
Heat Stability Tests for Test Beverages
Made with Native and Cheese WPI

| | Native Whey | Cheese Whey | Cheese Whey |
|---|---|---|---|
| | Protein | 1 Protein | 2 Protein |
| Heat Stability Test | Pass | Fail | Fail |
| Results | | | |
| (5% w/w Aqueous | | | |
| Protein Beverage | | | |
| at 90.5° C. for 20 min) | | | |

The test beverages made with native whey protein remained a flowable liquid without a significant increase in viscosity after the heat treatment and passed the heat stability test. In contrast, the test beverages made with cheese whey protein solidified into a hard gel by the end of the heat treatment and failed the heat stability test.

Comparing Exemplary Protein Beverages with Conventional Protein Waters

Protein profiles were taken for an exemplary protein-containing beverage and three comparative protein beverages. Selected characteristics of the protein-containing beverages are listed in Table 5 below:

TABLE 5
Characteristics of Protein-Containing Beverages Used in Protein Profiles:

| | | Comparative | Comparative | Comparative |
|---|---|---|---|---|
| | | Protein Beverage | Protein Beverage | Protein Beverage |
| | Exemplary Bev | #1 | #2 | #3 |
| Protein Source | Native Whey | Whey Protein | Whey Protein | Whey Protein |
| | Protein Isolate | Isolate | Isolate | Isolate |
| Protein/serving (g) | 20 | 20 | 22 | 20 |
| Sweetener | Organic Cane | Stevia Extract | Monk Fruit | Sucralose |
| | Sugar & Stevia | | Extract | |
| | Extract | | | |
| Acid | Phosphoric Acid | Phosphoric Acid | Phosphoric Acid | Phosphoric Acid |
| Colors | Vegetable | Not Added | Natural Color | Artificial Colors |
| | Concentrate | | | |
| | (Carrot & Sweet | | | |
| | Potato) | | | |
| Sodium Source | Sodium Citrate | Not Added | Sodium Citrate | Salt |
| Sodium/serving(mg) | 140 | 170 | 230 | 160 |
| Potassium | Not Added | Not Added | Potassium Citrate | Not Added |
| Source | | | | |
| Potassium/Serving | 110 | None Declared | 70 | 0 |
| (mg) | | | | |

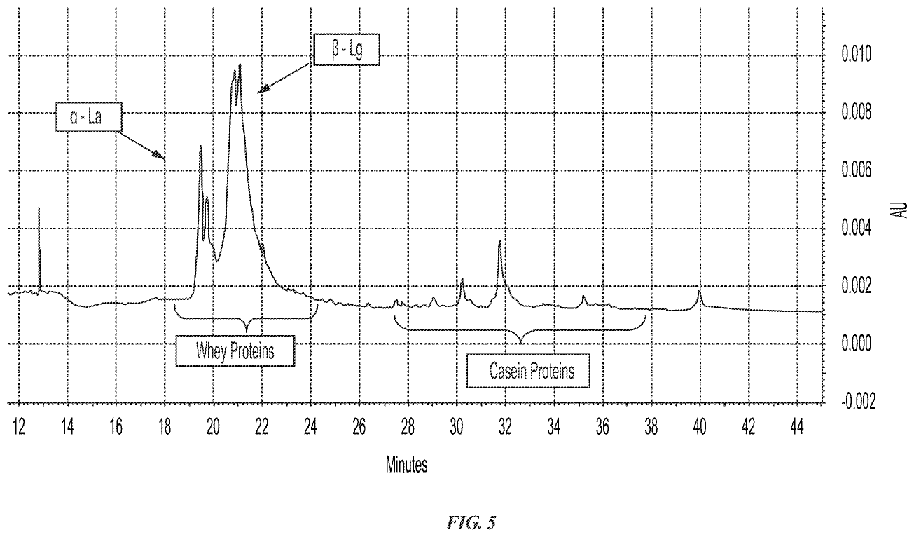

FIG. 5 shows a capillary electrophoresis protein profile of the exemplary protein-containing beverage. The beverage includes a native whey protein source as the sole protein source. The protein profile of FIG. 5 is similar to the protein profile in FIG. 2 for a native whey protein sample. Both protein profiles show large peaks for α-lactalbumin and β-lactoglobulin whey proteins, and significant peaks for intact casein proteins (e.g., α, β, and κ-caseins). Both protein profiles also show an absence of cGMPs.

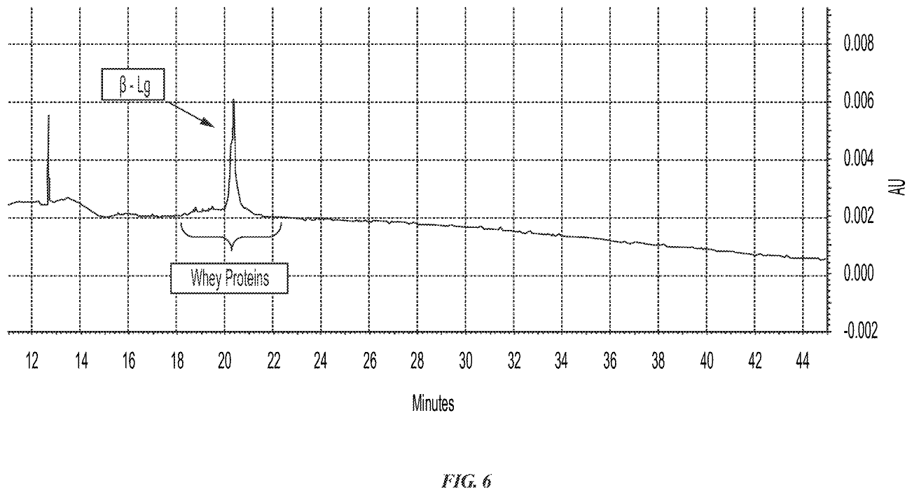

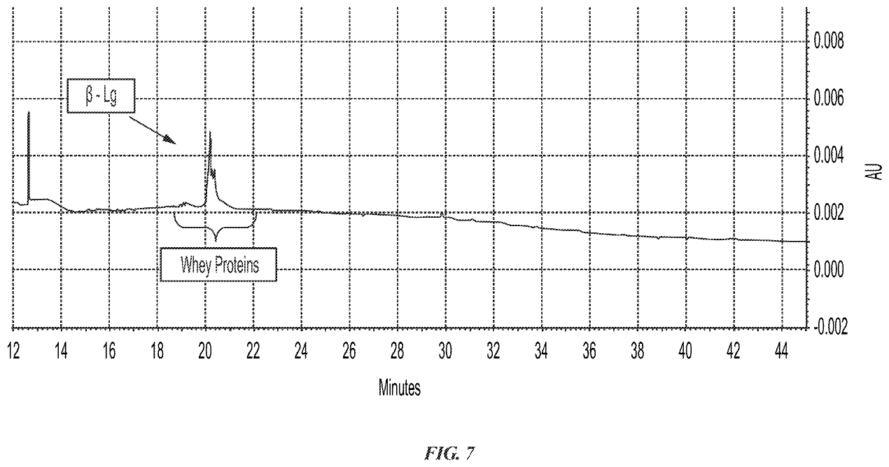

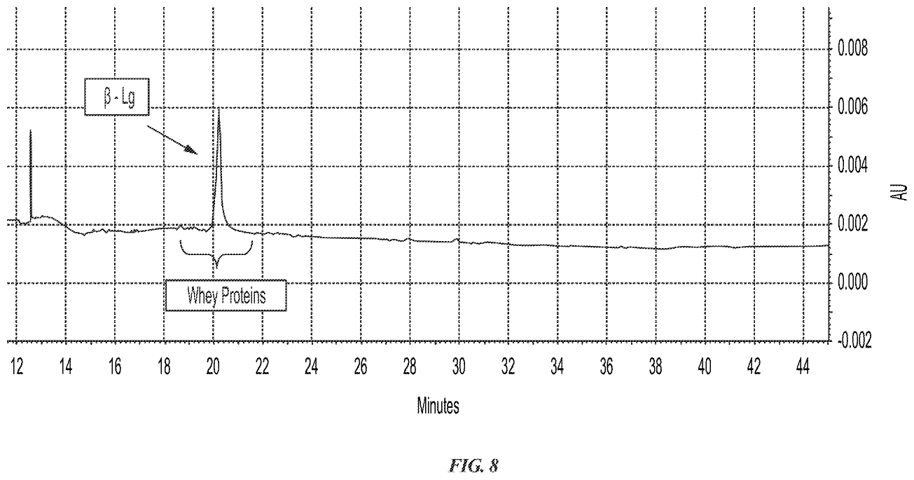

FIGS. 6-8 show capillary electrophoresis protein profiles for the three commercially-available protein waters (i.e., Comparative Protein Beverages #1, #2, #3). All three protein profiles show significant peaks for cGMPs and the absence of peaks for intact casein proteins. This is indicative of cheese whey protein as the primary protein source for these comparative proteins waters.

As noted above, the differences in the protein profiles between the exemplary beverage, made with native whey protein, and the comparative protein waters, made with cheese whey proteins, translate into differences in amino acid profiles. The branched-chain amino acid (BCAA) profiles for an Exemplary Protein-Beverage and three of the comparative protein waters (i.e., Comparative Protein Beverages #1, #2, and #3) were compared by measuring the weight percentage of the amino acids leucine, isoleucine, and valine for each beverage. Individual amino acids can be identified and quantified using high pressure liquid chromatography and ultra-violet detection (HPLC-UV). The results of the measurements are listed below in Table 6:

TABLE 6
BCAA Profiles for Exemplary and Comparative Protein Beverages

| | Exemplary Bev | Comp Bev #1 | Comp Bev #2 | Comp Bev #3 |
|---|---|---|---|---|
| Amino Acid | (wt.% protein) | (wt.% protein) | (wt.% protein) | (wt.% protein) |
| Leucine | 12.3 | 12.3 | 9.4 | 10.1 |
| Isoleucine | 5.4 | 5.2 | 5.7 | 6.1 |
| Valine | 5.1 | 4.7 | 5.2 | 5.3 |
| TOTAL | 22.8 | 22.2 | 20.3 | 21.5 |

Only Comparative Protein Beverage #1 had comparable levels of leucine as the Exemplary Protein Beverage, but even this protein water has a lower total amount of BCAAs than the Exemplary Protein Beverage.

The differences in the protein profiles between the exemplary beverage, made with native whey protein, and the comparative protein waters, made with cheese whey proteins, also translate into differences in their essential amino acid (EAA) profiles. The essential amino acid profiles for an Exemplary Protein-Beverage and three of the comparative protein waters (i.e., Comparative Protein Beverages #1, #2, and #3) were compared by measuring the weight percentage of the amino acids histidine, lysine, methionine, phenylalanine, threonine, and tryptophan, for each beverage. The results of the measurements are listed below in Table 7:

TABLE 7
EAA Profiles for Exemplary and Comparative Protein Beverages

| | Exemplary Bev | Comp Bev #1 | Comp Bev #2 | Comp Bev #3 |
|---|---|---|---|---|
| Amino Acid | (wt.% protein) | (wt.% protein) | (wt.% protein) | (wt.% protein) |
| Histidine | 0.4 | 0.4 | 0.4 | 0.4 |
| Isoleucine | 5.4 | 5.2 | 5.7 | 6.1 |
| Leucine | 12.3 | 12.3 | 9.4 | 10.1 |
| Lysine | 2.2 | 2.2 | 2.0 | 2.2 |
| Methionine | 0.4 | 0.4 | 0.4 | 0.4 |
| Phenylalanine | 0.8 | 0.7 | 0.6 | 0.5 |
| Threonine | 4.8 | 0.9 | 1.4 | 1.4 |
| Tryptophan | 0.5 | 0.4 | 0.4 | 0.4 |
| Valine | 5.1 | 4.7 | 5.2 | 5.3 |
| TOTAL | 31.9 | 27.3 | 25.5 | 26.5 |

Table 7 shows the Exemplary Protein Beverage had significantly higher total EEAs compared to all three Comparative Protein Beverages. The Exemplary Protein Beverage had an unusually high level of threonine, an essential amino acid that helps regulate protein in the body and is the precursor to serine and glycine. As a precursor for serine and glycine, threonine is also essential for collagen and elastin which is required for strong muscles and connective tissue (heart health). Threonine is also an essential building block for T-cells by the thymus gland which are critical for immune health. In the digestive tract, threonine is needed to produce the protective gel layer on the intestines and helps protect them from digestive enzymes. Animal studies have shown deficiency results in digestive issues and a reduction in immune response. The assumption is a disruption to the gut membrane reduces nutrient absorption and can lead to other health problems. Threonine along with methionine and aspartic acid helps support liver function for processing fats.

In general threonine is an essential amino acid to humans so it must be provided by the diet. It is an important amino acid in several major body functions including immune response through production of T-cells, digestive health through development of the mucus layer and protection of the intestine, liver health through the support of fat processing and for healthy muscles and connective tissue as the precursor for serine and glycine.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having disclosed several embodiments, it will be recognized by those of skill in the art that various modifications, alternative constructions, and equivalents may be used without departing from the spirit of the embodiments. Additionally, a number of well-known processes and elements have not been described in order to avoid unnecessarily obscuring the present technology. Accordingly, the above description should not be taken as limiting the scope of the technology.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included. Where multiple values are provided in a list, any range encompassing or based on any of those values is similarly specifically disclosed.

As used herein and in the appended claims, the singular forms “a”, “an”, and “the” include plural references unless the context clearly dictates otherwise. Thus, for example, reference to “a material” includes a plurality of such materials, and reference to “the cell” includes reference to one or more cells and equivalents thereof known to those skilled in the art, and so forth.

Also, the words “comprise(s)”, “comprising”, “contain(s)”, “containing”, “include(s)”, and “including”, when used in this specification and in the following claims, are intended to specify the presence of stated features, integers, components, or operations, but they do not preclude the presence or addition of one or more other features, integers, components, operations, acts, or groups.

What is claimed is:

1. A protein-containing beverage comprising:
water; and
2 wt. % to 8 wt. % protein based on total weight of the beverage, wherein the protein comprises:
whey protein, wherein the whey protein includes at least 12 wt. % leucine based on total weight of the whey protein; and
casein protein.

2. The protein-containing beverage of claim 1, wherein the whey protein is 50 wt. % to 99.9 wt. % of the protein.

3. The protein-containing beverage of claim 1, wherein the casein protein is selected from the group consisting of α-caseins, β-casein, and κ-caseins.

4. The protein-containing beverage of claim 1, wherein the casein protein remains in solution.

5. The protein-containing beverage of claim 1, wherein the casein protein is 0.1 wt. % to 50 wt. % of the protein.

6. The protein-containing beverage of claim 4, wherein the casein protein is 5 wt. % to 10 wt. % of the protein.

7. The protein-containing beverage of claim 1, wherein the protein-containing beverage has a whey protein to casein protein weight ratio ranging from 50:50 to 99.9:0.01.

8. The protein-containing beverage of claim 1, wherein the beverage further includes less than 0.45 wt. % sodium based on the total weight of the beverage.

9. The protein-containing beverage of claim 1, wherein the beverage further includes 0.02 wt. % to 3 wt. % potassium based on the total weight of the beverage.

10. The protein-containing beverage of claim 1, wherein the beverage further includes 0.4 wt. % to 1.65 wt. % calcium based on the total weight of the beverage.

11. The protein-containing beverage of claim 1, wherein the beverage contains no glycomacropeptides.

12. The protein-containing beverage of claim 1, wherein the beverage has a lactose content of 1 wt. % or less.

13. The protein-containing beverage of claim 1, wherein the beverage has a food-grade acid content of 2 wt. % or less.

14. The protein-containing beverage of claim 1, wherein the beverage has a viscosity of 5 cP or less at room temperature.

15. The protein-containing beverage of claim 1, wherein the protein is not derived from a cheesemaking process.

16. The protein-containing beverage of claim 1, wherein the protein containing beverage further comprises at least one ingredient selected from the group consisting of an acidification agent, a flavoring agent, a probiotic, a botanical, a fruit ingredient, a vegetable ingredient, caffeine, and collagen.

17. A method of making a protein-containing beverage, the method comprising:
filtering milk to form a protein isolate, wherein the protein isolate comprises:
whey protein, wherein the whey protein includes at least 12 wt. % leucine based on total weight of the whey protein; and
casein protein;
combing the protein isolate with an aqueous composition to form the protein-containing beverage.

18. The method of claim 17, wherein the whey protein is 50 wt. % to 99.9 wt. % of the protein isolate.

19. The method of claim 17, wherein the casein protein is selected from the group consisting of α-caseins, β-casein, and κ-caseins.

20. The method of claim 17, wherein the casein protein is 0.1 wt. % to 50 wt. % of the protein isolate.

21. The method of claim 17, wherein the casein protein is 5 wt. % to 10 wt. % of the protein.

22. The method of claim 17, wherein the protein-containing beverage further includes less than 0.45 wt. % sodium based on the total weight of the beverage.

23. The method of claim 17, wherein the protein-containing beverage further includes 0.02 wt. % to 3 wt. % potassium based on the total weight of the beverage.

24. The method of claim 17, wherein the protein-containing beverage has a viscosity of 5 cP or less at room temperature.

25. The method of claim 17, wherein the protein-containing beverage does not include any protein derived from a cheesemaking process.

26. A method of making a protein-containing beverage product, the method comprising:
providing a protein-containing aqueous mixture that includes total proteins comprising:
casein protein; and
whey protein, wherein the whey protein has at least 12 wt. % leucine based on a total weight of the whey protein;
homogenizing and pasteurizing the protein-containing aqueous mixture;
bottling the homogenized and pasteurized protein-containing aqueous mixture to form the protein-containing beverage product.

27. The method of claim 26, wherein the method further comprises adding a food-grade acid to the protein-containing aqueous mixture to adjust the mixture to a pH range of 2 to 4.

28. The method of claim 26, wherein the food-grade acid added to the protein-containing aqueous mixture adjusts its pH to about 3.0 to 3.5

29. The method of claim 27, wherein the food-grade acid comprises phosphoric acid.

30. The method of claim 26, wherein the whey protein is 50 wt. % to 99.9 wt. % of the total proteins.

31. The method of claim 26, wherein the casein protein is selected from the group consisting of α-caseins, β-casein, and κ-caseins.

32. The method of claim 26, wherein the casein protein is 0.1 wt. % to 50 wt. % of the total proteins.

33. The method of claim 32, wherein the casein protein is 5 wt. % to 10 wt. % of the protein.

34. The method of claim 26, wherein the protein-containing aqueous mixture further comprises at least one flavor agent, at least one color agent, and at least one sweetener.

35. The method of claim 26, wherein the protein-containing aqueous mixture further comprises at least one of sodium citrate, potassium citrate, or tricalcium citrate.

36. The method of claim 26, wherein none of the total proteins is derived from a cheesemaking process.

37. The method of claim 26, wherein the protein-containing beverage product contains no glycomacropeptides.

38. The method of claim 26, wherein the beverage has a lactose content of 1 wt. % or less.

39. The method of claim 26, wherein the whey protein comprises α-lactalbumin and β-lactoglobulin.

40. The method of claim 26, wherein the casein protein comprises β-casein.
